(19) United States
(12) Patent Application Publication
(10) Pub. No.: US 2013/0123612 A1
(43) Pub. Date: May 16, 2013

(54) MRT SURFACE COIL ARRAY

(71) Applicant: Jörg ROTHARD, Litzendorf (DE)

(72) Inventor: Jörg ROTHARD, Litzendorf (DE)

(21) Appl. No.: 13/464,922

(22) Filed: May 4, 2012

(30) Foreign Application Priority Data
May 6, 2011 (DE) 10 2011 075 452.0

(51) Int. Cl. A61B 5/055

(52) U.S. Cl. CPC A61B 5/055
USPC 600/422

(57) ABSTRACT

A surface coil for an imaging system includes a spine coil array that includes a plurality of coil elements. The surface coil also includes a cardiac coil array including a plurality of coil elements. The spine coil array and the cardiac coil array are both arranged in the same housing.

This application claims the benefit of DE 10 2011 075 452.0, filed on May 6, 2011.

### BACKGROUND

The present embodiments relate to a surface coil.

Magnetic resonance tomography machines (MRTs) for examining subjects or patients by magnetic resonance tomography are known, for example, from DE10314215B4.

In MR tomography, “surface coils” (or “loops”) may be used to acquire images with a high signal-to-noise ratio. With this system, the excited nuclei induce a voltage in the coil that is amplified by a low-noise pre-amplifier (LNA) and routed by cable to the receiver electronics at the MR frequency.

In order to improve the signal-to-noise ratio for high-resolution images, “high field” equipment is used. The basic field strengths of this equipment may be, for example, as much as 3 Tesla or even higher. Since it is often possible to connect more coil elements (loops) to an MR receiver system than there are receivers available, a switching matrix (e.g., an RCCS), for example, is fitted between the receive antennas and receivers. This routes the receive channels active at that instant to the available receivers, allowing more coil elements to be connected than there are receivers available. This is because for whole-body coverage, readouts are only needed from those coils that are located in the image acquisition area Field of View (FoV) and/or in the homogeneity volume of the MRT magnet.

The individual antenna elements are also referred to below as coil elements. The term “coil” or coil array is used, for example, to denote an antenna that may include one or more coil elements (array coil). A coil includes the coil elements, the preamplifier, additional electronics and cabling, the housing and may include a cable having a connector, via which the coil is connected to the system. The term “system” is understood, for example, to be the MR machine. “Parallel imaging techniques” (product name used by Siemens is iPAT) are used in MR to reduce the measurement time. These techniques utilize the spatial resolution of the individual receive coil elements in order to reduce the measurement time. The higher the number of coil elements on a given geometry, the greater the potential to use acceleration techniques. This is the motivation behind developing surface coils that have a large number of channels. The increasing number of channels of surface coils also provide (because the patient geometry remains the same) that the individual coil elements become smaller. Smaller coil elements deliver a higher SNR in the vicinity of the coil than larger elements. The increase in the number of channels in a surface coil therefore provides two advantages: better image quality in the vicinity of the receive antennas and better image quality overall when using iPAT.

If a large number of surface-coil elements are operated immediately adjacent, the surface-coil elements are decoupled from one another, because otherwise the coupling between the coil elements may result in increased noise in the image. Inductive and capacitive decoupling techniques are used for this purpose. Individual elements may be inductively decoupled, for example, by overlapping the individual elements or by transformers. In addition to the requirement for good decoupling, the elements of a spine coil are also arranged so that the elements may be used not only for abdominal imaging but also, for example, for imaging the spine. If, however, the coil is intended to be used for cardiac imaging, the array of coil elements has to meet fundamentally different requirements.

If a coil is larger than the FoV, it is advantageous to connect to receivers only those coil elements that actually lie in the FoV.

According to one solution, two different coil types are used for spinal imaging and cardiac imaging. In spine coils, four coil elements are arranged side-by-side in the x-direction and eight coil elements side-by-side in the z-direction. Dedicated coils are employed for cardiac imaging, during the use of which the spine coil is removed from the system. An example of this is a “32 channel cardiac array” from the company Invivo. In this case, 16 elements are located in the posterior section of the coil in a significantly smaller space than is covered by the spine coil.

### SUMMARY AND DESCRIPTION

The present embodiments may obviate one or more of the drawbacks or limitations in the related art. For example, a surface coil for an imaging system is optimized.

According to the present embodiments, a combination of two antenna configurations are provided within a coil housing.

For example, a spine coil array and a cardiac coil array may both be arranged in one/the same patient couch for a magnetic resonance tomography machine.

An antenna array optimized for spine imaging and an antenna array optimized for cardiac imaging may be arranged in two layers in the xz-plane (which is horizontal and/or orthogonal to the insertion direction z) at a minimum distance apart.

Either the spine antenna or the cardiac antenna may be used to receive the MR signals (e.g., by the switching of PIN diodes). Each of the antennas that are not being used for receiving may be disabled.

The cardiac antenna, which has a significantly higher channel density than the spine coil, may be implemented in the z-direction only in a specific area of the overall coil. The z-extent of the spine coil may be suitable for imaging the entire spinal column.

The total number of receive elements that may be connected to the MR system may be limited by the design of the coil connectors, via which the coil is connected to the system. If the total number of the receive elements of spine antenna and cardiac antenna exceeds the number of elements that may be connected to the MR system, an automatic switching system that connects either the receive elements of the spine antenna or the receive elements of the cardiac antenna to the MRT system may be used.

According to the present embodiments, a multipurpose antenna configuration array may be used as a spine coil (surface coil) and as a cardiac coil.

### BRIEF DESCRIPTION OF THE DRAWINGS

FIG. 1 shows a plan view of one embodiment of a surface coil;

FIG. 2 shows a longitudinal cross-section of one embodiment of a surface coil;

FIG. 3 shows a schematic diagram of an MRT system;

FIG. 4 shows one embodiment of low-noise preamplifiers (LNAs) and detuning circuits; and

FIG. 5 shows one embodiment of LNAs and detuning circuits.

### DETAILED DESCRIPTION OF THE DRAWINGS

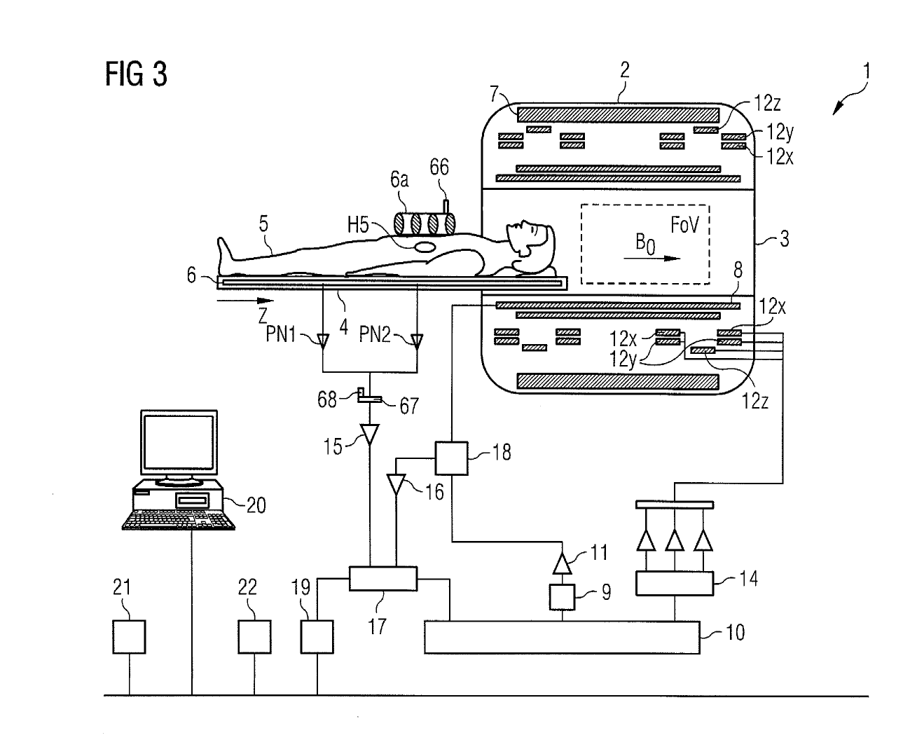

FIG. 3 shows a magnetic resonance machine MRT 1 intended for MRT imaging including a whole-body coil 2 having a chamber 3 that is approximately tubular in shape. A patient couch 4 carrying a body (e.g., of a patient 5 (including or excluding surface coil(s) 6 and/or 6a on and/or underneath the patient)) may be moved into the chamber 3 in the direction of the arrow z in order to generate recordings of the patient 5. A surface coil 6 that enables good recordings in a local area (e.g., the field of view (FoV)) is placed on the patient. Signals from the surface coil 6 may be analyzed (e.g., converted into images and stored or displayed) by an analysis unit (e.g., including elements 67, 66, 15, 17) of the MRT 1 that may be connected, for example, via cable (e.g., coaxial cable) and/or by radio communication (66, 68) to the surface coil 6 and/or 6A. A surface coil 6, W, H arranged in or on a patient couch 4 may also be provided as the surface coil.

In order to examine a body 5 (e.g., an examination subject or a patient) by magnetic resonance imaging using a magnetic resonance machine MRT 1, the body 5 is exposed to various magnetic fields that are matched to one another as closely as possible in terms of temporal and spatial characteristics. A strong magnet (e.g., a superconducting magnet 7) in a measuring enclosure having an aperture 3 that is tunnel-shaped, for example, produces a static, strong main magnetic field B₀ that equals, for example, 0.2 Tesla to 3 Tesla or higher. The body 5 to be examined is supported on the patient couch 4 and is moved into a region of the main magnetic field B0 that is approximately homogenous in the FoV. The nuclear spins of atomic nuclei of the body 5 are excited by magnetic RF excitation pulses that are emitted via an RF antenna shown in FIG. 3 in simplified form as a body coil 8 (and/or via at least one surface coil if applicable). RF excitation pulses are, for example, generated by a pulse generator 9 that is controlled by a pulse-sequence control unit 10. After amplification by an RF amplifier 11, the pulses are fed to the RF antenna 8. The RF system shown in FIG. 3 is merely represented schematically. In some embodiments, more than one pulse generator 9, more than one RF amplifier 11, and a plurality of RF antennas 8 are used in one magnetic resonance machine 1.

The magnetic resonance machine 1 has gradient coils 12x, 12 y, 12 z that are used during a measurement to produce magnetic gradient fields for selective slice excitation and for spatial encoding of the measurement signal. The gradient coils 12x, 12 y, 12 z are controlled by a gradient-coil control unit 14 that, like the pulse generator 9, is connected to the pulse-sequence control unit 10.

Signals emitted by excited nuclear spins are received by the body coil 8 and/or at least one surface coil 6, W, are amplified by assigned RF preamplifiers 16, 15 and processed and digitized by at least one receiver 17. The recorded measurement data is digitized and stored as complex numbers in a k-space matrix. An associated MR image may be reconstructed by a multidimensional Fourier transform from the k-space matrix containing the assigned values.

For a coil that may be operated both in transmit and receive mode such as, for example, the body coil 8 (and/or a surface coil 6, 6A), the correct signal routing is controlled by a transmit-receive switch 18 connected in series with the coil.

An image processing unit 19 generates, from the measurement data, an image that is displayed to a user via an operating console 20 and/or stored in a memory unit 21. A central processing unit 22 controls the individual system components.

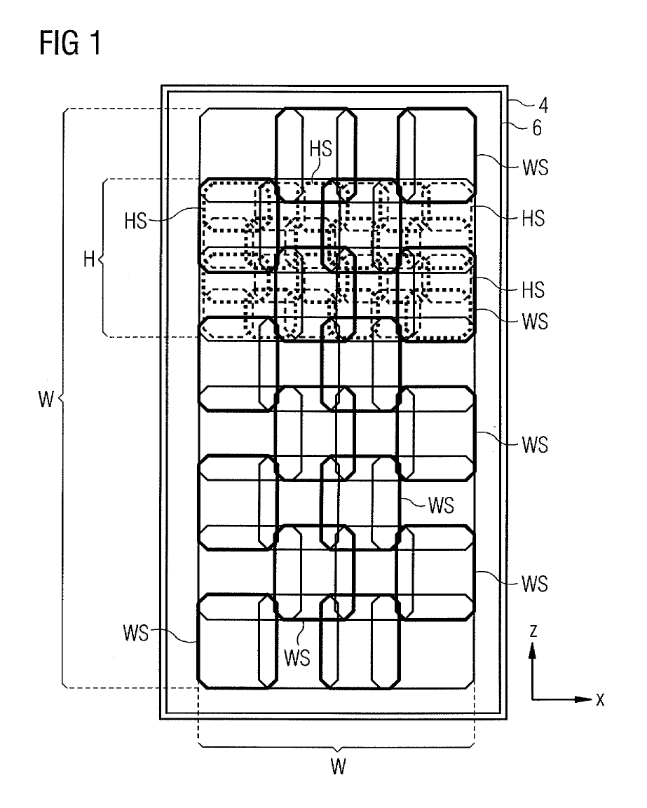

FIG. 1 shows, in plan view (e.g., arranged in a patient couch), one embodiment of a surface coil 6. The surface coil 6 includes a spine coil W (e.g., a spine antenna) including a plurality of coil elements WS. The plurality of coil elements WS is located within a spine examination region (e.g., distributed over (almost) the entire length of the patient couch 4 and over part of the width or over the entire width of the patient couch 4).

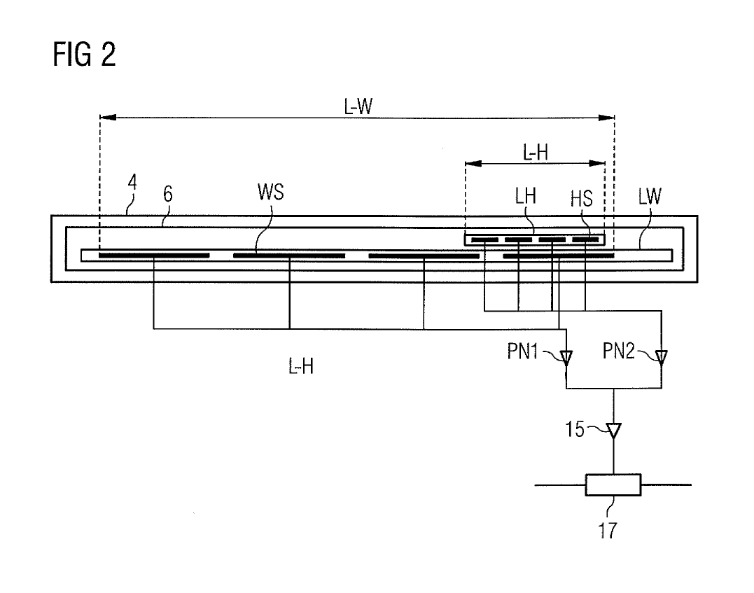

The surface coil 6 also includes a cardiac coil H including a plurality of coil elements HS in a cardiac examination region (reference sign L-H in FIG. 2).

The spine coil array W and the cardiac coil array H are arranged in the same housing (e.g., both are arranged in a patient couch, and/or both are arranged in a housing 6 in the patient couch 4). The coils/antennas optimized for spine imaging and the coils/antennas optimized for cardiac imaging are arranged with respect to each other in two layers (reference sign L-W in FIG. 2; for the coil elements WS of the spine coil W and L-H for the coil elements HS of the cardiac coil array H) in the xz-plane at the minimum possible separation between the layers (e.g., having a separation less than their thickness).

Within the cardiac coil array H, the density of coil elements HS (per surface area and/or per unit of length in the z-direction and/or per unit of length in the x-direction) may be greater than the density of coil elements WS outside the cardiac coil array H. “Density” of coil elements HS, WS may be the number of coil elements that are present per unit length and/or width and/or surface area.

In the region of the cardiac coil H, the coil elements HS (shown dotted in FIG. 1 to improve clarity) may be smaller than the coil elements WS (the latter (WS) being arranged at approximately the same density outside as inside the cardiac examination region H).

FIG. 2 shows in longitudinal cross-section a surface coil 6 according to the present embodiments that is, or may be, arranged in a patient couch 4. Signals received from the examination subject 5 from a plurality of coil elements HS and/or WS may be transferred by radio communication and/or via cable (e.g., for a plurality of coils, by time-multiplexing together in one cable).

Switching between reception in a processing device 17 of receive signals only from the coil elements WS of the spine coil W and of receive signals only from the coil elements HS of the cardiac coil H may be provided (e.g., by PIN diodes PIN1, PIN2 that are selectively switched (alternatively from each other) into the conducting state, or by more than two diodes or suitable switches).

If a coil array W, H is larger than the image acquisition area (FoV) of the MRT 1, then only those coil elements (WS and/or HS) that are actually lying in the FoV at a point in time or during an MRT recording (recording series) of a patient may be connected to receivers 17.

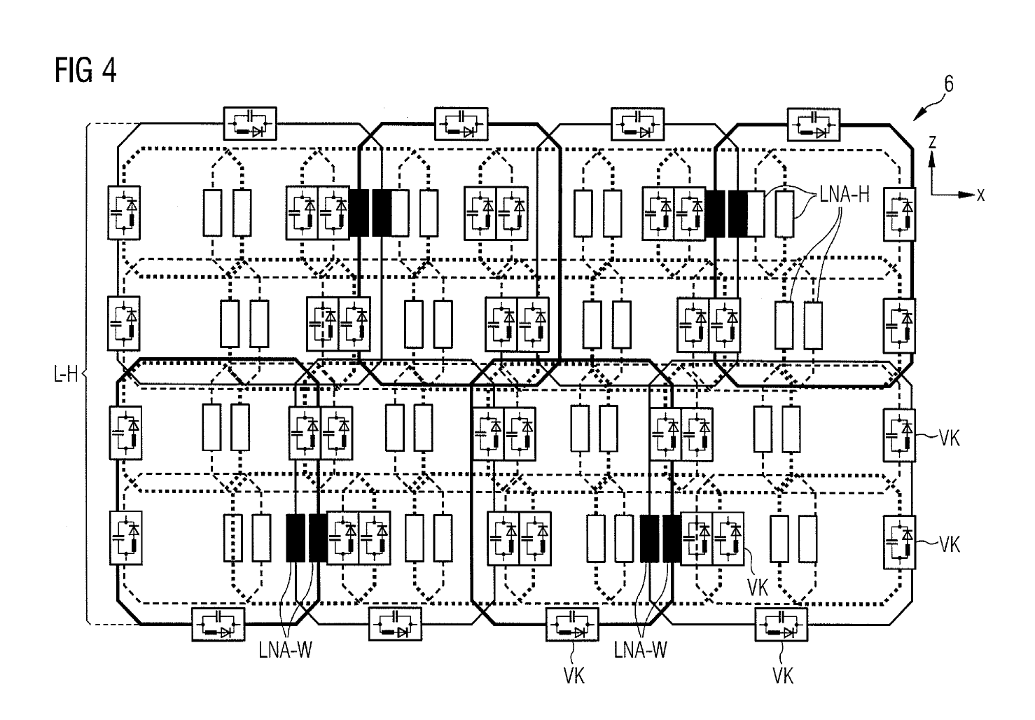

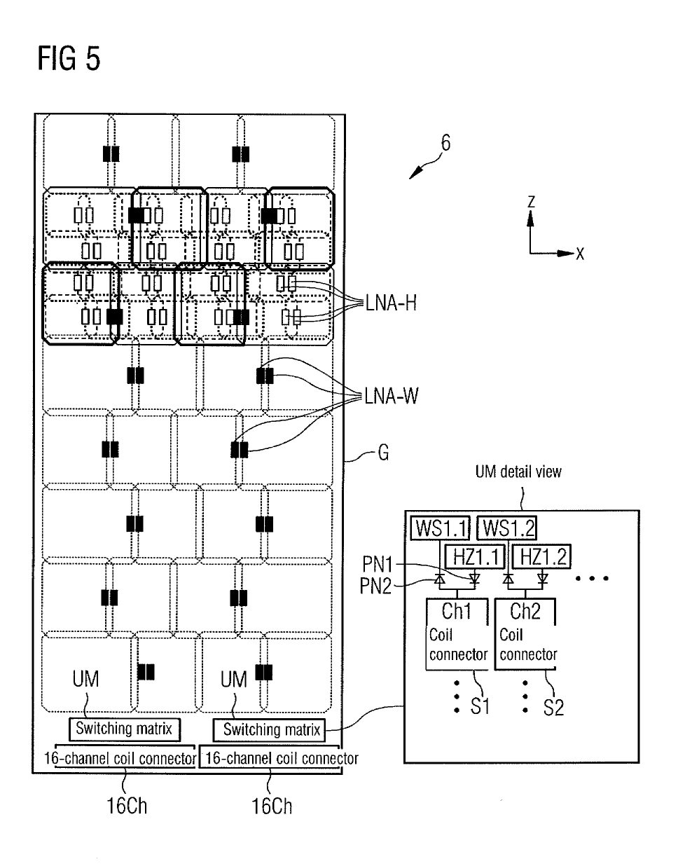

A connection of two antenna layouts within a coil housing according to the present embodiments may have the following advantages. There is no need to change the coil located on the patient couch when switching between the two types of examination (e.g., spine examination and cardiac examination). The resultant time-saving enables a higher patient throughput. Due to the two antenna layouts optimized for the particular application, both spinal and cardiac examinations may be carried out optimally using only one coil array despite the different requirements. FIGS. 4 and 5 illustrate some details of an exemplary embodiment of a surface-coil array 6.

FIG. 4 shows a segment of a region L-H (containing cardiac coils and spine coils) of a coil 6 from FIG. 2. In FIG. 4, LNA-H (see also the empty rectangles in FIG. 4) respectively denotes an LNA of the cardiac coil array H. Reference sign LNA-W (see also the blocked-in rectangles in FIG. 4) respectively denotes an LNA of the spine coil array W, reference sign G denotes a housing of the surface coil 6, and VK denotes each of the detuning circuits for decoupling the receive antenna from the transmit field and for decoupling the surface-coil elements from one another (e.g., may be used when there is a sufficient number of channels in each 16-channel coil connector containing 16 channels each).

When, for example, the number of channels of the entire surface coil 6 (including spine coil array W and cardiac coil array H) does not exceed the number of channels provided by the connectors, the detuning circuits (LC resonant circuits VK switched by PIN diodes) provided in the individual coil elements may also be used for isolating the two coil types (HS and WS). These detuning circuits VK are used in each receive coil to switch these coils into a transparent state for the RF field during the transmit phase.

If, however, there are more coil elements (HS, WS) present in the surface coil 6 than may be transferred through the connectors (e.g., S1, S2) to the (MRT) system, then, as shown in FIG. 5, an additional electronics unit (e.g., switching matrix UM; UM also shown as a magnified detail in the bottom right of FIG. 5) is provided for selecting between two or more individual elements (e.g., WS1.1 or HZ1.1 connected to channel “CH1 coil connector;” either WS1.2 or HZ1.2 connected to channel “CH2 coil connector”) of the surface coil 6 for connection to a receive channel 17 (e.g., that may be connected to a coil connector CH1 of a coil connector socket 16Ch for 16 channels) of the MRT 1.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A surface coil for an imaging system, the surface coil comprising:
a spine coil array comprising a first plurality of coil elements; and
a cardiac coil array comprising a second plurality of coil elements,
wherein the spine coil array and the cardiac coil array are both arranged in a housing.

2. The surface coil as claimed in claim 1, wherein the spine coil array and the cardiac coil array are both arranged in a patient couch for a magnetic resonance tomography (MRT) machine.

3. The surface coil as claimed in claim 1, wherein a region of the heart of a patient is positionable in a region above the cardiac coil array.

4. The surface coil as claimed in claim 1, wherein a spinal region of a patient is positionable in a region vertically above the spine coil array.

5. The surface coil as claimed in claim 1, wherein the spine coil array is an array operable to acquire an image of a spine or of a sub-region of the spine of an examination subject.

6. The surface coil as claimed in claim 1, wherein the cardiac coil array is an array operable to acquire an image of the heart of an examination subject.

7. The surface coil as claimed in claim 1, wherein the spine coil array and the cardiac coil array are each arranged within separate layers.

8. The surface coil as claimed in claim 1, wherein the spine coil array and the cardiac coil array are arranged within layers that are parallel to each other.

9. The surface coil as claimed in claim 1, further comprising a switching device,
wherein depending on a switch-setting of the switching device, either only coils of the spine coil array or only coils of the cardiac coil array are selectively connected to an analysis unit for signals received from the examination subject.

10. The surface coil as claimed in claim 9, wherein at a point in time, only the coil array that is not being used at the point in time is disabled, is not connected to an analysis unit for signals received from the examination subject, or a combination thereof.

11. The surface coil as claimed in claim 1, wherein the density of the coil elements of the first plurality of coil elements is greater than the density of the coil elements of the second plurality of coil elements.

12. The surface coil as claimed in claim 1, wherein the cardiac coil array has a density of the coil elements that is more than twice as great as a density of the coil elements of the spine coil array.

13. The surface coil as claimed in claim 1, wherein the cardiac coil array has a density of the coil elements that is more than three times as great as a density of the coil elements of the spine coil array.

14. The surface coil as claimed in claim 1, wherein a density of the coil elements of the cardiac coil array is constant in a region of the cardiac coil array, a density of the coil elements of the spine coil array is constant in a region of the spine coil array, or a combination thereof.

15. The surface coil as claimed in claim 1, wherein the spine coil array is suitable for imaging the entire spine of an examination subject, is at least 180 cm, or a combination thereof.

16. The surface coil as claimed in claim 1, wherein the length of the cardiac coil array is less than a third of the spine coil array in the z-direction.

17. The surface coil as claimed in claim 2, wherein the second plurality of the coil elements are located only in a region of the patient couch that is closer to the MRT machine in an insertion direction of the patient couch, or in a region that is one half or one third of the patient couch.

18. The surface coil as claimed in claim 1, wherein the surface coil is a magnetic resonance tomography (MRT) surface coil.

19. The surface coil as claimed in claim 9, wherein the switching device comprises PIN diodes.

20. The surface coil as claimed in claim 12, wherein the density of the coil elements of the cardiac coil array is more than twice as great as the density of the coil elements of the spine coil in the z-direction.

21. The surface coil as claimed in claim 1, wherein the density of the coil elements of the cardiac coil array is more than three times as great as the density of the coil elements of the spine coil array in the z-direction.

22. The surface coil as claimed in claim 15, wherein the spine coil array is at least 190 cm.

23. The surface coil as claimed in claim 15, wherein the spine coil array is at least 200 cm.

24. The surface coil as claimed in claim 16, wherein the length of the cardiac coil array is less than a quarter of the spine coil array in the z-direction.

25. The surface coil as claimed in claim 16, wherein the length of the cardiac coil array is less than a fifth of the length of the spine coil array in the z-direction.
